(19) United States
(12) Patent Application Publication
(10) Pub. No.: US 2022/0226226 A1
(43) Pub. Date: Jul. 21, 2022

(54) PERSONAL CARE SPRAY COMPOSITIONS AND METHODS COMPRISING TRANS-1-CHLORO-3,3,3-TRIFLUOROPROPENE

(71) Applicant: HONEYWELL INTERNATIONAL INC., Charlotte, NC (US)

(72) Inventors: Anthony Anzalone, Charlotte, NC (US); Yiu Keung Ling, Charlotte, NC (US); Jon Thomas Herdlein, Charlotte, NC (US); Leonard Michael Stachura, Charlotte, NC (US)

(73) Assignee: HONEYWELL INTERNATIONAL INC., Charlotte, NC (US)

(21) Appl. No.: 17/672,589

(22) Filed: Feb. 15, 2022

Related U.S. Application Data
(62) Division of application No. 16/555,428, filed on Aug. 29, 2019.
(60) Provisional application No. 62/725,492, filed on Aug. 31, 2018.

(51) Int. Cl. A61K 8/73; A61Q 5/06; A61Q 15/00; A61K 8/26; A61K 8/04; A61K 8/34; A61Q 5/00; A61K 8/31; A61K 8/72

(52) U.S. Cl. CPC A61K 8/732; A61Q 5/06; A61Q 15/00; A61K 8/26; A61K 8/72; A61K 8/34; A61Q 5/008; A61K 8/315; A61K 8/046

(57) ABSTRACT

Disclosed are methods of applying a leave-on personal care treatment products comprising:
a) providing a container having a nozzle or opening for selectively releasing the contents thereof:
b) including in the container a leave-on personal care formulation comprising:
   (i) an active component for affecting the personal care of the user;
   (ii) a carrier for the active component, said carrier comprising at least about 50% by weight of trans-HCFO-1233zd and optionally a co-carrier in amounts of up to about 50% by weight; and
c) spraying said leave-on personal care formulation from the container and onto the area of the user to be treated.

### CROSS REFERENCES TO RELATED APPLICATIONS

The present application is a divisional of U.S. patent application Ser. No. 16/555,428 filed Aug. 29, 2019, now pending, which is related to and claims priority to U.S. Provisional Application 62/725,492, filed Aug. 31, 2018. The present application is also related to and claims priority as a continuation in part of U.S. application Ser. No. 16/113,451 filed on Aug. 27, 2018, now pending, which in turn claims priority as a divisional to U.S. application Ser. No. 15/288,398 filed on Oct. 7, 2016, now U.S. Pat. No. 10,059,849, which in turn is a divisional to U.S. application Ser. No. 13/593,391 filed on Aug. 23, 2012, now U.S. Pat. No. 9,485,986, which in turn claims priority to Provisional Application Ser. No. 61/526,859, filed on Aug. 24, 2011. The contents of all applications identified in the paragraph are incorporated herein by reference.

### BACKGROUND

Many personal care products are applied in the form of a spray, including hair sprays, dry shampoos, deodorants and antiperspirants. Such products generally comprise an active component or components that provide the intended function when it applied to the skin, hair or scalp. Such products also generally include carriers which solvate, disperse or emulsify the active ingredient so that it can be effectively transported frorn its container to the surface of the user to be treated. In many applications the container includes a propellant or other means to propel the active/carrier to the surface to be treated in the form of a spray. As a result of the application of the composition in the intended manner, the active component becomes operative upon or in association with the removal, usually by evaporation, of the solvent or carrier.

Generally, it is desired for such personal care products to deliver the active ingredient in the form of a well-formed spray that dries quickly from the skin, hair or scalp of the user. It has also become increasing desirable for the carrier to have a relatively small negative environmental impact, such as having a low ozone depletion potential, a low global warming potential and low volatile organics content. In addition to favorable environmental properties, the carrier preferably also has, depending on the particular application, an inertness with respect to the active ingredients), low toxicity and low flammability, among other properties.

One carrier that has heretofore been commonly used in such applications is ethanol. Applicants have conic to appreciate that important and unexpected improvements in the performance of such products can be achieved by the use of novel carrier in a spray-applied personal care formulation that is capable of providing a well-formed spray that is quick drying and environmentally friendly.

### BRIEF DESCRIPTION OF THE DRAWING

FIG. 1 is a schematic view of a aerosol spray product according to one embodiment of the present invention.

FIG. 2 is a chart depicting the experimental results reported in Examples 1 and 2.

FIG. 3 is a series of photographs depicting the spray pattern results from Example 3.

### SUMMARY

The present invention provides compositions, methods and products which comprise one or more active components and which utilize as a carrier trans-1-chloro-3,3,3-trifluoropropene (trans-HCFO-1233zd).

The present invention includes personal care products comprising:
a) container having a nozzle or opening for releasing the contents thereof;
b) a personal care formulation in the container comprising:
   (i) an active component for affecting the personal care of the user; and
   (ii) a carrier for the active component, said carrier comprising at least about 50% by weight based on the weight of the carrier of trans-HCFO-1233zd and optionally a co-carrier in amounts of up to about 50% by weight based on the weight of the carrier.

The present invention also includes a personal care formulation comprising:
(i) an active component for affecting the personal care of a user of the formulation; and
(ii) a carrier for the active component, said carrier comprising at least about 50% by weight based on the weight of the carrier of trans-HCFO-1233zd and optionally a co-carrier in amounts of up to about 50% by weight based on the weight of the carrier.

The present invention includes a method of applying a personal care product to a user comprising:
a) providing a container having a opening or nozzle for expelling or releasing at least a portion of contents therefrom;
b) including in said container a personal care formulation comprising:
   (i) an active component for affecting the personal care of the user; and
   (ii) a carrier for the active component, said carrier comprising at least about 50% by weight of trans-HCFO-1233zd based on the weight of the carrier and optionally a co-carrier in amounts of up to about 50% by weight based on the weight of the carrier; and
c) removing at least a portion of said personal care formulation from the container and applying it onto the area of the user to receive the personal care product.

Applicants have unexpectedly found that the preferred compositions, methods, and products of the present invention provide not only highly favorable and desirable environmental properties, including preferably low GWP, low ODP and/or low VOC, the trans-HCFO-1233zd described herein for use as the carrier possess a desirable but difficult to achieve mosaic of other properties, including providing the formulation, methods and products with a quick drying ability and at the same time providing one or more of the following difficult to achieve properties in combination with the quick drying effect:
substantial inertness with respect to the active ingredient(s),
low toxicity,
low flammability.
good spray forming ability,
good ability to uniformly carry the active component in the amounts required.

Applicants have unexpectedly found that the preferred compositions, products and methods of the present invention have the unexpected advantage of being quick drying, that is, the carrier is readily and quickly removed after application to the user by evaporation. In highly preferred embodiments, the formulations, products and methods are quick-drying and self drying. As used herein, the term “self-drying” refers to the removal of the carrier after it is applied by simply being exposed to the body of the user in the environment of use without the introduction of any separate drying source, and preferably with little or no additional steps after the spraying step. Thus, in preferred compositions, methods and products, the time required to dry the formulation is relatively low and requires relatively little, and preferably no additional energy after the application step.

The present invention includes a sprayable leave-on treatment product comprising:
a) container having a spray nozzle for expelling the contents thereof;
b) a personal care formulation in the container comprising:
   (i) an active component for affecting the cleanliness, and/or appearance and/or feel of the hair and/or scalp and/skin of the user;
   (ii) a carrier for the active component, said carrier comprising at least about 50% by weight based on the weight of the carrier of trans-HCFO-1233zd and optionally a co-carrier in amounts of up to about 50% by weight based on the weight of the carrier; and
   iii) propellant in the container for propelling at least a portion of said personal care formulation from said container through said spray nozzle.
      As used here, the term “leave-on treatment” refers to products and formulations which are applied to the user and are not then intended to be rinsed or washed off as part of the treatment process. Such materials are also sometimes referred to in the industry as “dry” compositions or products since a water wash or rinse is not required. In preferred embodiments according to this paragraph, the propellant comprises, consists essentially of, or consists of one or more of trans1234ze, HFC-152a, dimethyl ether (DME), HFC-152a, dimethyl ether (DME), a hydrocarbon propellant, including blends of hydrocarbon propellants, such as blends of propane, isobutane and butane (an example of such a blend being A46) and combinations of two or more of these.

The present invention also includes a sprayable leave-on treatment formulation comprising:
(i) an active component affecting the cleanliness, and/or appearance and/or feel of the hair and/or scalp and/or skin of the user; and
(ii) a carrier for the active component, said carrier comprising at least about 50% by weight of trans-HCFO-1233zd based on the weight of the carrier and optionally a co-carrier in amounts of up to about 50% by weight based on the weight of the carrier.

The present invention includes a method of applying a leave-on treatment product to a user comprising:
a) providing a container having a spray nozzle for expelling at least a portion of contents therefrom;
b) including in said container a leave-on treatment formulation comprising:
   (i) an active component affecting the cleanliness, and/or appearance and/or feel of the hair and/or scalp and/or skin of the user; and
   (ii) a carrier for the active component, said carrier comprising at least about 50% by weight of trans-HCFO-1233zd based on the weight of the carrier and optionally a co-carrier in amounts of up to about 50% by weight based on the weight of the carrier; and
c) spraying said personal care formulation from the container and onto the area of the user to receive the personal care product.

### DETAILED DESCRIPTION OF THE DISCLOSURE

The present invention provides compositions, methods and products which comprise one or more active components and which utilize as a carrier trans-1-chloro-3,3,3-trifluoropropene (trans-HCFO-1233zd).

The present application is utilized to great advantage in connection with personal care compositions, methods and products and in particular such compositions, methods and products that are intended to be sprayed and left-on after application. By way of example, the present invention can be used in connection with products such as hair sprays, dry shampoos, sun screen sprays, antiperspirants, diaper rash products and similar personal care products.

For hair spray products, the active component can include in preferred embodiments can include polymers and other components that enhance the shine, feel, hold or the like of the hair.

For deodorant and antiperspirant products, the active component can include in preferred embodiments inorganics/aluminum slats and the like.

For dry shampoo products, the active component can include personal care starches, that is, starches that are used to absorb oils and/or other contaminants contained on the hair and/or scalp and/or skin. In preferred embodiments, the active component of the personal care product comprises, consists essentially of or consist of a personal care starch. In preferred embodiments, the personal care starch comprises, consists essentially of or consist of one or more of: rice starch, tapioca starch, corn starch, aluminum starch and talc-starch.

By way of example, starches of the type sold by Akzo-Noble under the trade designations Natsorb HFB and Dry-flo PC can be used. The active component, and in particular active component comprising personal care starch, may also include magnesium stearate NF.

The present invention includes a sprayable personal care product comprising:
a) container having a spray nozzle for expelling the contents thereof; and
b) a personal care formulation in the container comprising:
   (i) an active component for affecting the personal care of the user; and
   (ii) a carrier for the active component, said carrier comprising at least about 50% by weight based on the weight of the carrier of trans-HCFO-1233zd and optionally a co-carrier in amounts of up to about 50% by weight based on the weight of the carrier; and
   (iii) propellant in the container for propelling said personal care formulation from said container through said spray nozzle. In preferred embodiments according to this paragraph, the propellant comprises, consists essentially of, or consists of one or more of trans1234ze, HFC-152a, dimethyl ether (DME), HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable personal care product comprising:
a) container having a spray nozzle for expelling the contents thereof; and
b) a personal care formulation in the container comprising:
   (i) an active component in an amount effective to affect the personal care of the user;
   (ii) from about 10% by weight to about 50% by weight based on the contents of the container of a carrier for the active component, said carrier comprising at least about 50% by weight based on the weight of the carrier of trans-HCFO-1233zd and optionally a co-carrier in amounts of up to about 50% by weight based on the weight of the carrier; and
   (iii) at least about 40% by weight of propellant in the container based on the contents of the container for propelling said personal care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable personal care product comprising:
a) container having a spray nozzle for expelling the contents thereof; and
b) a personal care formulation in the container comprising:
   (i) an active component in an amount effective to affect the personal care of the user;
   (ii) from about 10% by weight to about 40% by weight based on the contents of the container of a carrier for the active component, said carrier comprising at least about 50% by weight based on the weight of the carrier of trans-HCFO-1233zd and optionally a co-carrier in amounts of up to about 50% by weight based on the weight of the carrier; and
   (iii) at least about 70% by weight of propellant in the container based on the contents of the container for propelling said personal care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

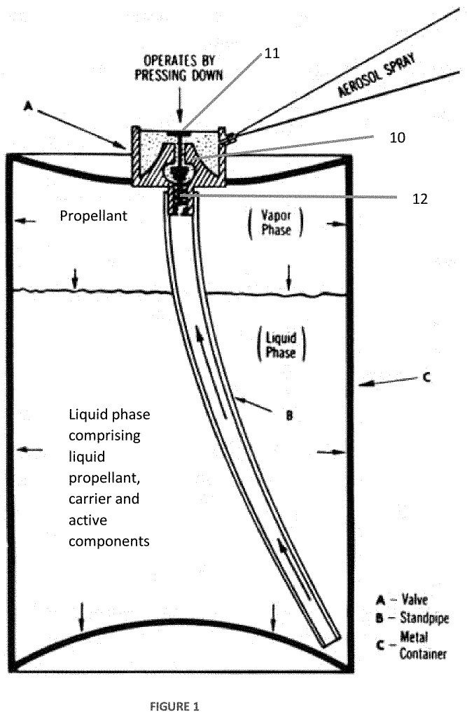

An exemplary aerosol spray device is disclosed in FIG. 1 hereof. As illustrated in the Figure, a container (typically a metal canister capable of holding fluid under pressure) has an opening closed with a spray nozzle A. The nozzle A includes a valve portion 10 and an actuator 11. A vapor space occupied by the gaseous portion of the propellant exerts a pressure on the liquid components of the personal care composition. When the actuator 11 is depressed by the user against the closing force of the spring 12, the valve is opened and the pressure exerted by the propellant forces the liquid through the dip-tube B and out of the valve in the form of an aerosol spray. When the user releases the actuator button 11, the spring reseats the valve and the flow of fluid is discontinued.

The present invention also includes a sprayable personal care formulation comprising:
(i) an active component for affecting the personal care of a user of the formulation; and
(ii) a carrier for the active component, said carrier comprising at least about 50% by weight based on the weight of the carrier of trans-HCFO-1233zd and optionally a co-carrier in amounts of up to about 50% by weight based on the weight of the carrier.

The present invention also includes a sprayable personal care formulation comprising:
(i) an active component in an amount effective to affect the personal care of the user;
(ii) from about 10% by weight to about 40% by weight based on the total weight of the formulation of a carrier for the active component, said carrier comprising at least about 50% by weight based on the weight of the carrier of trans-HCFO-1233d and optionally a co-carrier in amounts of up to about 50% by weight based on the weight of the carrier; and
(iii) at least about 40% by weight based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable personal care formulation comprising:
(i) an active component in an amount effective to affect the personal care of the user;
(ii) from about 10% by weight to about 40% by weight based on the total weight of the formulation of a carrier for the active component, said carrier comprising at least about 50% by weight based on the weight of the carrier of trans-HCFO-1233zd and optionally a co-carrier in amounts of up to about 50% by weight based on the weight of the carrier; and
(iii) at least about 70% by weight based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a personal care product to a user comprising:
a) providing a container having a spray nozzle for expelling at least a portion of contents therefrom;
b) including in said container a personal care formulation comprising:
   (i) an active component for affecting the personal care of the user; and
   (ii) a carrier for the active component, said carrier comprising at least about 50% by weight based on the weight of the carrier of trans-HCFO-1233zd and optionally a co-carrier in amounts of up to about 50% by weight based on the weight of the carrier; and
c) spraying said personal care formulation from the container and onto the area of the user to receive the personal care product.

The present invention includes a method of applying a personal care product to a user comprising:
a) providing a container having a spray nozzle for expelling the contents thereof; and
b) including in said container a personal care formulation comprising:
   (i) an active component in an amount effective to affect the personal care of the user;
   (ii) from about 10% by weight to about 40% by weight based on the contents of the container of a carrier for the active component, said carrier comprising at least about 50% by weight based on the weight of the carrier of trans-HCFO-1233zd and optionally a co-carrier in amounts of up to about 50% by weight based on the weight of the carrier; and
   (iii) at least about 40% by weight of propellant in the container based on the contents of the container for propelling said personal care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a personal care product to a user comprising:
a) providing a container having a spray nozzle for expelling the contents thereof; and
b) including in said container a personal care formulation comprising:
   (i) an active component in an amount effective to affect the personal care of the user;
   (ii) from about 10% by weight to about 40% by weight based on the contents of the container of a carrier for the active component, said carrier comprising at least about 50% by weight based on the weight of the carrier of trans-HCFO-1233zd and optionally a co-carrier in amounts of up to about 50% by weight based on the weight of the carrier; and
   (iii) at least about 70% by weight of propellant in the container based on the contents of the container for propelling said personal care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising at least about 50% by weight based on the weight of the carrier of trans-HCFO-1233zd and optionally a co-carrier in amounts of up to about 50% by weight based on the weight of the carrier; and
   (iii) propellant in the container for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable hair care formulation comprising:
(i) an active component in an amount effective to affect the personal care of the user;
(ii) from about 10% by weight to about 40% by weight based on the contents of the container of carrier for the active component, said carrier comprising at least about 50% by weight of trans-HCFO-1233zd based on the weight of the carrier and optionally a co-carrier in amounts of up to about 50% by weight based on the weight of the carrier; and
(iii) at least about 40% based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable hair care formulation comprising:
(i) an active component in an amount effective to affect the personal care of the user;
(ii) from about 10% by weight to about 40% by weight based on the contents of the container of carrier for the active component, said carrier comprising at least about 50% by weight of trans-HCFO-1233zd based on the weight of the carrier and optionally a co-carrier in amounts of up to about 50% by weight based on the weight of the carrier; and
(iii) at least about 70% based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user; and
   (ii) a carrier for the active component, said carrier comprising at least about 50% by weight of trans-HCFO-1233zd based on the weight of the carrier and optionally a co-carrier in amounts of up to about 50% by weight based on the weight of the carrier; and
c) spraying said personal care formulation from the container and onto the area of the user to receive the personal care product.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user; and
   (ii) a carrier for the active component, said carrier comprising at least about 50% by weight of trans-HCFO-4233zd based on the weight of the carrier and optionally a co-carrier in amounts of up to about 50% by weight based on the weight of the carrier;
   (iii) at least about 40% based on the total weight of the contents of the container of propellant; and
c) spraying said personal care formulation from the container and onto the area of the user to receive the personal care product. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user; and
   (ii) a carrier for the active component, said carrier comprising at least about 50% by weight of trans-HCFO-1233zd based on the weight of the carrier and optionally a co-carrier in amounts of up to about 50% by weight based on the weight of the carrier;
   (iii) at least about 70% based on the total weight of the contents of the container of propellant; and
c) spraying said personal care formulation from the container and onto the area of the user to receive the personal care product. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising at least about 50% by weight based on the weight of the carrier of trans-HCFO-1233zd and optionally a co-carrier in amounts of up to about 50% by weight based on the weight of the carrier, said co-carrier comprising a C1-C3 alcohol; and
   (iii) propellant in the container for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising at least about 50% by weight based on the weight of the carrier of trans-HCFO-1233zd and optionally a co-carrier in amounts of up to about 50% by weight based on the weight of the carrier, said co-carrier comprising a C1-C3 alcohol; and
   (iii)) at least about 40% K based on the total weight of the contents of the container of propellant for propelling said hair care formulation from said container through said spray nozzle In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising at least about 50% by weight based on the weight of the carrier of trans-HCFO-1233zd and optionally a co-carrier in amounts of up to about 50% by weight based on the weight of the carrier, said co-carrier comprising a C1-C3 alcohol; and
   (iii)) at least about 70% based on the total weight of the contents of the container of propellant for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising at least about 50% by weight based on the weight of the carrier of trans-HCFO-2337d and optionally a co-carrier in amounts of up to about 50% by weight based on the weight of the carrier, said co-carrier comprising a C1-C3 alcohol; and
   (iii) propellant in the container for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable hair care formulation comprising:
(i) an active component for affecting the personal care of a user of the formulation at least by absorbing oil from the hair or scalp of the user; and
(ii) a carrier for the active component, said carrier comprising at least about 50% by weight based on the weight of the carrier of trans-HCFO-1233zd and optionally a co-carrier in amounts of up to about 50% by weight based on the weight of the carrier, said co-carrier comprising a C1-C3 alcohol.

The present invention also includes a sprayable hair care formulation comprising:
i) an active component for affecting the personal care of a user of the formulation at least by absorbing oil from the hair or scalp of the user;
(ii) a carrier for the active component, said carrier comprising at least about 50% by weight based on the weight of the carrier of trans-HCFO-1233zd and optionally a co-carrier in amounts of up to about 50% by weight based on the weight of the carrier, said co-carrier comprising a C1-C3 alcohol; and
(iii) at least about 40% based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable hair care formulation comprising:
(i) an active component for affecting the personal care of a user of the formulation at least by absorbing oil from the hair or scalp of the user;
(ii) a carrier for the active component, said carrier comprising at least about 50% by weight based on the weight of the carrier of trans-HCFO-1233zd and optionally a co-carrier in amounts of up to about 50% by weight based on the weight of the carrier, said co-carrier comprising a C1-C3 alcohol; and
(iii) at least about 70% based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user; and
   (ii) a carrier for the active component, said carrier comprising at least about 50% by weight based on the weight of the carrier of trans-HCFO-1233zd and optionally a co-carrier in amounts of up to about 50% by weight based on the weight of the carrier, said co-carrier comprising a C1-C3 alcohol; and
c) spraying said personal care formulation from the container and onto the area of the user to receive the personal care product.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user; and
   (ii) a carrier for the active component, said carrier solvating, emulsifying and/or dispersing said oil-absorbing active component and comprising at least about 50% by weight based on the weight of the carrier of trans-HCFO-1233zd and optionally a co-carrier in amounts of up to about 50% by weight based on the weight of the carrier; and
c) spraying said personal care formulation from the container and onto the area of the user to receive the personal care product.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising from about 50% by weight to about 90% by weight of trans-HCFO-1233zd and a co-carrier in amounts of from about 10% up to about 50% by weight, said co-carrier preferably comprising a C1-C3 alcohol, and preferably ethanol; and
   (iii) propellant in the container for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising from about 50% by weight to about 90% by weight of trans-HCFO-1233zd and a co-carrier in amounts of from about 10% up to about 50% by weight, said co-carrier preferably comprising a C1-C3 alcohol, and preferably ethanol; and
   (iii) at least about 40% based on the total weight of the contents of the container of propellant for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user;
   (ii) a carrier for the oil-absorbing active component, said carrier solvating, emulsifying and/or dispersing said oil-absorbing active component in the container and comprising from about 50% by weight to about 90% by weight of trans-HCFO-1233zd and a co-carrier in amounts of from about 10% up to about 50% by weight, said co-carrier preferably comprising a C1-C3 alcohol, and preferably ethanol; and
   (iii) at least about 70% based on the total weight of the contents of the container of propellant for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable hair care formulation comprising:
(i) an active component for affecting the personal care of a user of the formulation at least by absorbing oil from the hair or scalp of the user; and
(ii) a carrier for the active component, said carrier solvating, emulsifying and/or dispersing said oil-absorbing active component and comprising from about 50% by weight to about 90% by weight based on the weight of the carrier of trans-HCFO-1233zd and a co-carrier in amounts of from about 10% up to about 50% by weight based on the weight of the carrier, said co-carrier preferably comprising a C1-C3 alcohol, and preferably ethanol.

The present invention also includes a sprayable hair care formulation comprising:
(i) an active component for affecting the personal care of a user of the formulation at least by absorbing oil from the hair or scalp of the user;
(ii) a carrier for the active component, said carrier solvating, emulsifying and/or dispersing said oil-absorbing active component and comprising from about 50% by weight to about 90% by weight based on the weight of the carrier of trans-HCFO-1233zd and a co-carrier in amounts of from about 10% up to about 50% by weight based on the weight of the carrier, said co-carrier preferably comprising a C1-C3 alcohol, and preferably ethanol; and
(iii) at least about 40% based on the total weight of the contents of the container of propellant for propelling said hair care formulation from said container through said spray nozzle. In preferred embodiments according to this paragraph the propellant comprises, consists essentially of, or consist of one or more of trans1234ze, HFC-152a, dimethyl ether (DME) and A46.

The present invention also includes a sprayable hair care formulation comprising:
(i) an active component for affecting the personal care of a user of the formulation at least by absorbing oil from the hair or scalp of the user;
(ii) a carrier for the active component, said carrier solvating, emulsifying and/or dispersing said oil-absorbing active component and comprising from about 50% by weight to about 90% by weight based on the weight of the carrier of trans-HCFO-1233zd and a co-carrier in amounts of from about 10% up to about 50% by weight based on the weight of the carrier, said co-carrier preferably comprising a C1-C3 alcohol, and preferably ethanol; and
(iii) at least about 70% based on the total weight of the contents of the container of propellant for propelling said hair care formulation from said container through said spray nozzle. In preferred embodiments according to this paragraph the propellant comprises, consists essentially of, or consist of one or more of trans1234ze, HFC-152a, dimethyl ether (DME) and A46.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user; and
   (ii) a carrier for the active component, said carrier solvating, emulsifying and/or dispersing said oil-absorbing active component and comprising from about 50% by weight to about 90% by weight based on the weight of the carrier of trans-HCFO-1233zd and a co-carrier in amounts of from about 10% up to about 50% by weight based on the weight of the carrier, said co-carrier preferably comprising a C1-C3 alcohol, and preferably ethanol; and
c) spraying said personal care formulation from the container and onto the area of the user to receive the personal care product.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user;
   (ii) a carrier for the active component, said carrier solvating, emulsifying and/or dispersing said oil-absorbing active component and comprising from about 50% by weight to about 90% by weight based on the weight of the carrier of trans-HCFO-1233zd and a co-carrier in amounts of from about 10% up to about 50% by weight based on the weight of the carrier, said co-carrier preferably comprising a C1-C3 alcohol, and preferably ethanol; and
   (iii) at least about 40% based on the total weight of the contents of the container of propellant for propelling said hair care formulation from said container through said spray nozzle; and
c) spraying said personal care formulation from the container and onto the area of the user to receive the personal care product. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user;
   (ii) a carrier for the active component, said carrier solvating, emulsifying and/or dispersing said oil-absorbing active component and comprising from about 50% by weight to about 90% by weight based on the weight of the carrier of trans-HCFO-1233zd and a co-carrier in amounts of from about 10% up to about 50% by weight based on the weight of the carrier, said co-carrier preferably comprising a C1-C3 alcohol, and preferably ethanol; and
   (iii) at least about 70% based on the total weight of the contents of the container of propellant for propelling said hair care formulation from said container through said spray nozzle; and
c) spraying said personal care formulation from the container and onto the area of the user to receive the personal care product. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user, said active component comprising starch;
   (ii) a carrier for the oil-absorbing active component, said carrier solvating, emulsifying and/or dispersing said oil-absorbing active component in the container and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol; and
   (iii) propellant in the container for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane tan example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user, said active component comprising starch;
   (ii) a carrier for the oil-absorbing active component, said carrier solvating, emulsifying and/or dispersing said oil-absorbing active component in the container and comprising from about 50% by weight to about 75% by weight based on the weight of the carrier of trans-HCFO-1233zd and a co-carrier in amounts of from about 25% up to about 50% K by weight based on the weight of the carrier, said co-carrier preferably comprising a C1-C3 alcohol, and preferably ethanol; and
   (iii) at least about 40% based on the total weight of the contents of the container of propellant for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a sprayable dry hair care product comprising:
a) container having a spray nozzle for expelling the contents thereof;
b) a hair care formulation in the container comprising:
   (i) an active component for absorbing oil from the hair or scalp of the user, said active component comprising starch;
   (ii) a carrier for the oil-absorbing active component, said carrier solvating, emulsifying and/or dispersing said oil-absorbing active component in the container and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol; and
   (iii) at least about 70% based on the total weight of the contents of the container of propellant for propelling said hair care formulation from said container through said spray nozzle. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable hair care formulation comprising:
(i) an active component for affecting the personal care of a user of the formulation at least by absorbing oil from the hair or scalp of the user, said active component comprising starch; and
(ii) a carrier for the active component, said carrier solvating, emulsifying and/or dispersing said oil-absorbing active component and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol.

The present invention also includes a sprayable hair care formulation comprising:
(i) an active component for affecting the personal care of a user of the formulation at least by absorbing oil from the hair or scalp of the user, said active component comprising starch; and
(ii) a carrier for the active component, said carrier solvating, emulsifying and/or dispersing said oil-absorbing active component and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol; and
(iii) at least about 40% based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention also includes a sprayable hair care formulation comprising:
(i) an active component for affecting the personal care of a user of the formulation at least by absorbing oil from the hair or scalp of the user, said active component comprising starch; and
(ii) a carrier for the active component, said carrier solvating, emulsifying and/or dispersing said oil-absorbing active component and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol; and
(iii) at least about 70% based on the total weight of the formulation of propellant. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user, said active component comprising starch; and
   (ii) a carrier for the active component, said carrier solvating, emulsifying and/or dispersing said oil-absorbing active component and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol; and
c) spraying said personal care formulation from the container and onto the area of the user to receive the personal care product.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user, said active component comprising starch;
   (ii) a carrier for the active component, said carrier solvating, emulsifying and/or dispersing said oil-absorbing active component and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol; and
   (iii) at least about 40% based on the total weight of the contents of the container of propellant; and
c) spraying said personal care formulation from the container and onto the area of the user to receive the personal care product. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

The present invention includes a method of applying a hair care product to a user comprising:
a) providing a container having a spray nozzle for expelling at least a portion of contents therefrom;
b) including in said container a personal care formulation comprising:
   (i) an active component for affecting the personal care of the user by absorbing oil from the hair or scalp of the user, said active component comprising starch;
   (ii) a carrier for the active component, said carrier solvating, emulsifying and/or dispersing said oil-absorbing active component and comprising from about 50% by weight to about 75% by weight based on the weight of the carrier of trans-HCFO-1233zd and a co-carrier in amounts of from about 25% up to about 50% by weight based on the weight of the carrier, said co-carrier preferably comprising a C1-C3 alcohol, and preferably ethanol; and
   (iii) at least about 70% based on the total weight of the contents of the container of propellant; and
c) spraying said personal care formulation from the container and onto the area of the user to receive the personal care product. In preferred embodiments according to this paragraph, the propellant comprises, consists essentially of, or consists of one or more of trans1234ze, HFC-152a, dimethyl ether (DME), a hydrocarbon propellant, including blends of hydrocarbon propellants, such as blends of propane, isobutene and butane (an example of such a blend being A46) and combinations of two or more of these.

### EXAMPLES

### Example 1

Unless otherwise noted, the quantity indications below are understood as percentages by weight.

The formulas indicated in Table 1 below are provided by mixing the indicated components, with the exception of the propellant, and introducing them into an aerosol spray can. The aerosol cans were sealed with the valve/nozzle arrangement, and then the indicated propellant was added through the valve. The formulation was then applied to a sheet of wax paper by spraying the formulation from the can for one (1) second while the nozzle is held 6 inches from the paper under ambient conditions (about 73° F. and 66 relative humidity with a 61° F. dew point). The time from the completion of the one second spray to the paper is dry is recorded as drying time. For this purpose of determining when the paper is dry according to this measurement, the process is video recorded and the time to disappearance of a wet spot is determined and checked against the weight of the paper gain corresponding to a plateau in the weight gain of the paper to determine dry time. In other words, the paper is considered dry when the carrier portion of the formulation has substantially fully evaporated from the paper.

TABLE 1

| | FORMULATION | | | | |
|---|---|---|---|---|---|
| | 1A | 1B | 1C | 1D | 1E |
| Components, | | | | | |
| wt % | | | | | |
| Active | | | | | |
| Components | | | | | |
| Starch* | 5 | 5 | 5 | 5 | 5 |
| Carrier | | | | | |
| Ethanol | 15 | 11.25 | 7.5 | 3.75 | 0 |
| trans1233zd | 0 | 3.75 | 7.5 | 11.25 | 15 |
| Propellant (100% | 80 | 80 | 80 | 80 | 80 |
| trans1234ze) | | | | | |
| RESULTS | | | | | |
| Wt % Ethanol in | 100 | 75 | 50 | 25 | 0 |
| Carrier | | | | | |
| Wt % 1233zd in | 0 | 25 | 50 | 75 | 100 |
| carrier | | | | | |
| Wt. Ratio | NA | 3:1 | 1:1 | 1:3 | NA |
| Ethanol:1233zd | | | | | |
| Dry Time, seconds | 460 | 350 | 130 | 45 | <1 |
| Dry Time | 0 | 24 | 72 | 90 | 0 |
| Improvement, | | | | | |
| Relative % | | | | | |
| compared to | | | | | |
| propellant = 100% | | | | | |
| ethanol | | | | | |

*The starch component is an oil-absorbing starch of the type commonly used in the cosmetics industry, including those disclosed in the present specification and combinations of these.

### Example 2

Example 1 is repeated except that the propellant A46 (consisting of 15.2% propane and 84.8% isobutane producing a vapor pressure of 46 psig at 70° F.) was used instead of a propellant consisting of trans1234ze. The formulations tested and the results obtained are reported in Table 2 below:

TABLE 2

| | FORMULATION | | | | |
|---|---|---|---|---|---|
| | 2A | 2B | 2C | 2D | 2E |
| Components, | | | | | |
| wt % | | | | | |
| Active | | | | | |
| Components | | | | | |
| Starch* | 5 | 5 | 5 | 5 | 5 |
| Carrier | | | | | |
| Ethanol | 15 | 11.25 | 7.5 | 3.75 | 0 |
| trans1233zd | 0 | 3.75 | 7.5 | 11.25 | 15 |
| Propellant (100% | 80 | 80 | 80 | 80 | 80 |
| A46) | | | | | |
| RESULTS | | | | | |
| Wt % Ethanol in | 100 | 75 | 50 | 25 | 0 |
| Carrier | | | | | |
| Wt % 1233zd in | 0 | 25 | 50 | 75 | 100 |
| carrier | | | | | |
| Wt. Ratio | NA | 3:1 | 1:1 | 1:3 | NA |
| Ethanol:1233zd | | | | | |
| Dry Time, seconds | 300 | 270 | 51 | 25 | <1 |
| Dry Time | 0 | 10 | 83.3 | 92 | 0 |
| Improvement, | | | | | |
| Relative % | | | | | |
| compared to | | | | | |
| propellant = 100% | | | | | |
| ethanol | | | | | |

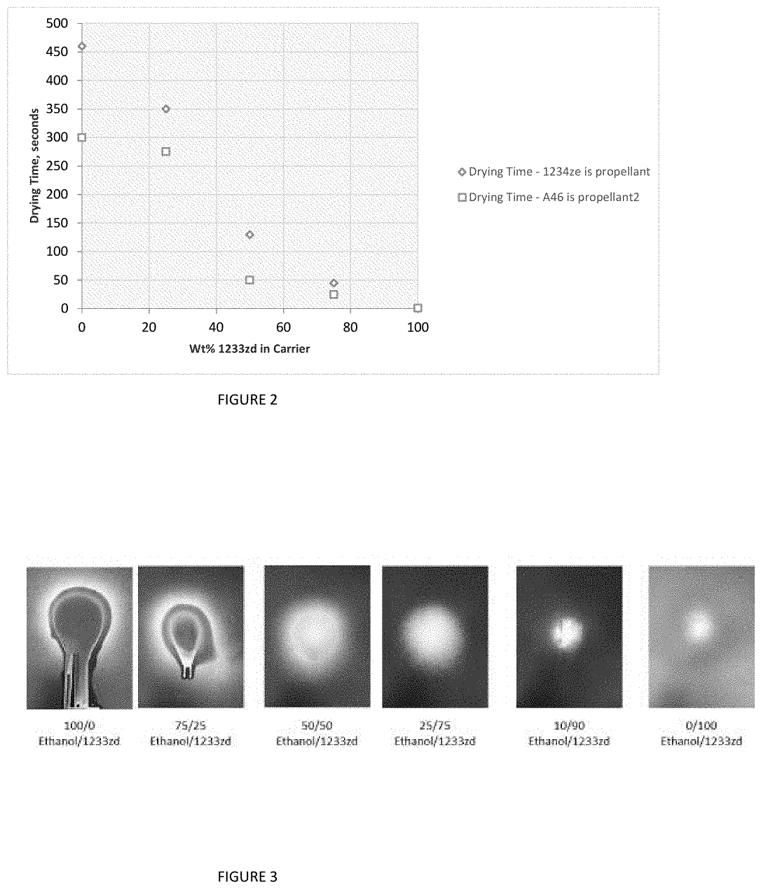

The results from Examples 1 and 2 are plotted in the chart provided as FIG. 2.

As can be seen from the results of the test work reported in Examples 1 and 2 and illustrated in FIG. 2, the dry times for formulations in which ethanol is 100% of the carrier produce dry times of from about 300 seconds to about 450 seconds. Importantly, using the same concentration of carrier in the formulation but with a carrier that is 75% ethanol and 25% trans1233zd produces a drying time improvement of from about 10 to about 25 relative percent. Importantly and surprisingly, a more substantial improvement in drying time occurs as the amount of trans1233zd in the carrier increases above 25% to about 75%, with drying times exhibiting the highest level of improvement with concentrations of trans1233zd in the carrier of from about 50% to 100%. For example, when A46 is the propellant, the drying time is reduced to about 50 seconds or less when the concentration of trans1233zd is about 50% or greater, which is an improvement 8 times more significant that that achieved when the concentration of trans1233zd in increases from zero to 25%. A similar dramatic and unexpected relative improvement occurs with trans1234ze as the propellant.

### Example 3

Unless otherwise noted, the quantity indications below are understood as percentages by weight.

The formulas indicated in Table 3 below are provided by mixing the indicated components, with the exception of the propellant, and introducing them into an aerosol spray can. The aerosol cans were sealed with the valve/nozzle arrangement, and then the indicated propellant was added through the valve. The formulation was then applied to a sheet of black construction paper by spraying the formulation from the can for five (5) seconds while the nozzle is held 12 inches from the paper under ambient conditions (about 73° F. and 66 relative humidity with a 61F dew point). Photographs of the spray pattern that is created with each formulation are provided in FIG. 3.

TABLE 3

| | FORMULATION | | | | | |
|---|---|---|---|---|---|---|
| | 3A | 3B | 3C | 3D | 3E | 3F |
| Components, | | | | | | |
| wt % | | | | | | |
| Active | | | | | | |
| Components | | | | | | |
| Starch* | 7 | 7 | 7 | 7 | 7 | 7 |
| Carrier | | | | | | |
| Ethanol | 37.0 | 19.43 | 18.5 | 6.48 | 3.7 | 0 |
| 1233zd | 0 | 6.48 | 18.5 | 19.43 | 33.3 | 37.0 |
| Propellant | | | | | | |
| trans1234ze | 56 | 56 | 56 | 56 | 56 | 56 |
| RESULTS | | | | | | |
| Wt % Ethanol in | 100 | 75 | 50 | 25 | 10 | 0 |
| Carrier | | | | | | |
| Wt % 1233zd in | 0 | 25 | 50 | 75 | 90 | 100 |
| carrier | | | | | | |

*The starch component is an oil-absorbing starch of the type commonly used in the cosmetics industry, including those disclose herein and combinations of these.

The results of Example 3 demonstrate a further unexpected advantage of the preferred formulations, products and methods of the present invention. In particular, it can be seen from FIG. 3 that the use of carrier consisting of ethanol and carrier consisting of 75% ethanol and 25% trans1233zd each produces a spray pattern that is much wetter than desirable for many applications, as can be seen by the high level of dripping from the formulation sprayed onto the paper. Unexpectedly, a 50/50 wt % blend of 1233zd and ethanol produces a highly desirable wide, uniformly distributed spray pattern of starch on the paper with no dripping. Similarly, results are shown for carriers having 25% ethanol and 75% trans1233zd and 10% ethanol and 90% trans1233zd.

What is claimed is:

1. A method of applying a leave-on personal care treatment product to an area of a user to be treated comprising:
a) providing a container having a nozzle or opening for selectively releasing the contents thereof;
b) including in the container a leave-on personal care formulation comprising:
(i) an active component for affecting the personal care of the user;
(ii) a carrier for the active component, said carrier comprising at least about 50% by weight of trans-HCFO-1233zd and optionally a co-carrier in amounts of up to about 50% by weight; and
c) spraying said leave-on personal care formulation from the container and onto the area of the user to be treated, wherein the dry-time after said spraying step is at least about 70% shorter than the dry time using said same steps a), b) and c) with said same components in said same amounts but in which the carrier consists of ethanol in the same weight as said carrier of (ii).

2. The method of claim 1 wherein said leave-on personal care treatment product comprises one or more of hair spray, dry shampoo, sun screen spray, antiperspirant, and diaper rash product.

3. The method of claim 1 wherein said leave-on personal care treatment product comprises hair spray and wherein said active component comprises a polymer and/or other components that enhance the shine and/or feel and/or hold of the hair.

4. The method of claim 1 wherein said leave-on personal care treatment product comprises antiperspirant and wherein said active component comprises an aluminum salts.

5. The method of claim 1 wherein said leave-on personal care treatment product comprises dry shampoo and wherein said active component comprises starch which absorb oils and/or other contaminants contained on the hair and/or scalp.

6. The method of claim 1 wherein said co-carrier is present in said carrier and comprises a C1-C3 alcohol.

7. The method of claim 1 wherein said co-carrier is present in said carrier and comprises ethanol.

8. The method of claim 1 wherein said carrier consists essentially of from about 50% by weight to about 90% by weight of trans-HCFO-1233zd and from about 10% to about 50% of ethanol.

9. A sprayable dry hair care product comprising:
a) container having a spray nozzle for expelling the contents thereof;
b) a hair care formulation in the container comprising:
(i) an active component for affecting the personal care of a user of the formulation at least by absorbing oil and/or other contaminants from the hair and/or scalp of the user; and
(ii) a carrier for the active component, said carrier solvating, emulsifying and/or dispersing said starch in the container and comprising from about 50% by weight to about 90% by weight based on the weight of the carrier of trans-HCFO-1233zd and from about 10% up to about 50% by weight based on the weight of the carrier of a C1-C3 alcohol; and
(iii) propellant in the container for propelling said hair care formulation from said container through said spray nozzle.

10. A sprayable hair care formulation comprising:
(i) starch for absorbing oil and/or other contaminants from the hair and/or scalp of the user; and
(ii) a carrier for the starch, said carrier solvating, emulsifying and/or dispersing said starch and comprising from about 50% by weight to about 75% by weight of trans-HCFO-1233zd and from about 25% up to about 50% by weight of a C1-C3 alcohol.
